(19) United States
(12) Patent Application Publication
(10) Pub. No.: US 2021/0282542 A1
(43) Pub. Date: Sep. 16, 2021

(54) Sterile, Disposable Toothbrush Having a Toothpaste Composition Bonded to Bristles for a Subject with a Compromised Immune System

(71) Applicant: Steven ZAKEN, Brentwood, NY (US)

(72) Inventor: Steven ZAKEN, Brentwood, NY (US)

(21) Appl. No.: 17/197,798

(22) Filed: Mar. 10, 2021

Related U.S. Application Data
(60) Provisional application No. 62/987,493, filed on Mar. 10, 2020.

(51) Int. Cl. A46B 11/00; A46B 9/04; A46D 1/00; A61C 19/06; A61C 19/08; A61K 8/21; A61K 31/085; A61Q 11/00; A61P 23/02

(52) U.S. Cl. CPC A46B 11/0003; A46B 9/04; A46D 1/0207; A46D 1/0292; A61C 19/063; A61K 2800/30; A61K 8/21; A61K 31/085; A61Q 11/00; A61P 23/02; A46B 2200/1066; A61C 19/08

(57) ABSTRACT

A sterile, disposable pre-pasted toothbrush having a toothpaste composition containing an anti-cavity agent and a numbing agent, with the toothpaste composition distributed over extra soft bristles and arranged on a small bristle head, with the toothbrush and toothpaste composition sterilized by an ozone treatment, to produce a disposable toothbrush particularly suited for patients with compromised immune systems.

This application claims priority to U.S. Provisional Patent Application No. 62/987,493, filed on Mar. 10, 2020, which is herein incorporated by reference in its entirety.

### FIELD OF THE INVENTION

The present invention is generally directed to the field of disposable, single-use pre-pasted toothbrushes for a subject with a compromised immune system.

### BACKGROUND OF THE INVENTION

To accommodate the busy and often harried traveler, disposable toothbrushes have been designed that can be used away from home, for example on a trip, or that can be offered for single use in establishments receiving the public, such as hotels and restaurants. These disposable toothbrushes have eliminated the need for a separate tube of toothpaste gel or other similar material by incorporating in or on the brush a sufficient dose of a flavored material for brushing the teeth, the flavor being added to encourage oral hygiene practices.

As used herein, the term “toothpaste” or “paste” refers to any suitable material for brushing one's teeth, including but not limited to toothpaste or tooth gel.

Many such prior art disposable toothbrushes are complex and/or expensive, as where a mechanism is included for distributing the toothpaste onto the bristles just prior to use. In other prior art disposable toothbrushes, the toothpaste is applied in advance or “preapplied” to the bristles, but such toothbrushes have proved unstable, in that the flavoring dissipates after a relatively short period of time, which is impractical as commercial considerations require a reasonable shelf life. As a result, prior art disposable toothbrushes have, for the most part, not proved commercially successful, primarily because no known prior art disposable toothbrush has satisfied the multiple criteria of a preapplied toothpaste thereby eliminating the need for a distribution mechanism, a reasonable shelf life, and a low manufacturing cost.

Other preapplied toothpaste formulations such as those described U.S. Pat. Nos. 5,605,756, 5,783,249, and 5,888,578, all of which are herein incorporated by reference in their entireties, implement a metal ion graft initiator and peroxide catalyst. Generally the amount and types of metal ions and peroxide catalysts used are safe for application in the mouth. However, a formulation omitting these ingredients would be preferable.

Disposable toothbrushes are also often used in hospitals and other care facilities. Proper oral care is essential for patients with a compromised immune system, such as those undergoing chemotherapy, far more so than for non-patients. It is recommended that such patients gently brush their teeth and tongue within 30 minutes after eating. However, oral care can be a quite painful process for patients receiving systemic chemotherapy and/or local radiotherapy, because these patients often develop painful ulcers of the oral mucosa and they have extreme sensitivity in their mouths. These patients are also at a particular risk of developing an opportunistic infection and require additional care on maintaining the sterile conditions of oral care tools to avoid further infections.

Thus, there is a need for an individually wrapped, sterile, disposable toothbrush having a composition bonded to the bristles of the toothbrush, which, when being used in oral care for a subject with a compromised immune system, has the benefits of relieving pain or touch sensitivity, being less intrusive and provide improved comfort, and being sterile to reduce further infections in the subject.

### SUMMARY OF THE INVENTION

In view of the above, an individually wrapped, inexpensive disposable toothbrush containing a toothpaste or gel composition having an anti-cavity agent (e.g., fluoride such as sodium fluoride) and a numbing agent (e.g., eugenol) for relieving pain or touch sensitivity, wherein the composition is bonded to the bristles of the toothbrush, is provided to a subject with a compromised immune system. The toothbrush bristles are small in dimension to fit in a compact head and are made extra soft for less painful, less intrusive, and more effective brushing suitable for use by patients with a compromised immune system, such as chemotherapy cancer patients. The disposable toothbrush and toothpaste composition undergo a sterilization process that lends the toothbrush particularly suited for the needs of patients with a compromised immune system. The disposable toothbrush and toothpaste composition are packed in a sealed package wrapping surrounding the toothbrush and toothpaste composition to provide airtight sealing and maintain the toothbrush and toothpaste composition in a sterile condition.

In addition, the disposable toothbrush has a toothpaste composition distributed over the bristles of the toothbrush without the use of metal graft initiators and catalysts, and in which the flavoring does not dissipate over a reasonable shelf life is provided.

One aspect of the invention relates to a sterilized toothbrush and toothpaste composition for a subject with a compromised immune system, comprising: a toothbrush having bristles comprising a substrate; and a toothpaste composition bonded to the substrate of the toothbrush bristles in the absence of a metal graft initiator, peroxide catalyst, or a combination thereof.

The toothpaste composition includes the flavored toothpaste formulation discussed in the embodiments below relating to the methods of and compositions for bonding toothpaste formulations to bristles of a toothbrush in the absence of a metal graft initiator, peroxide catalyst, or a combination thereof. The toothpaste composition also comprises an anti-cavity agent and a numbing agent in an amount sufficient to relieve pain or touch sensitivity in the subject with a compromised immune system. The toothbrush and toothpaste composition are sterilized with an ozone treatment.

Another aspect of the invention relates to a method of manufacturing a sterilized toothbrush and toothpaste composition for a subject with a compromised immune system, comprising: providing a toothbrush having bristles comprising a substrate; bonding a toothpaste composition to the substrate of the toothbrush bristles in the absence of a metal graft initiator, peroxide catalyst, or a combination thereof; and sterilizing the toothbrush and toothpaste composition.

As discussed in the above aspect of the invention, the toothpaste composition includes the flavored toothpaste formulation discussed in the embodiments below relating to the methods of and compositions for bonding toothpaste formulations to bristles of a toothbrush in the absence of a metal graft initiator, peroxide catalyst, or a combination thereof. The toothpaste composition also comprises an anti-cavity agent and a numbing agent in an amount sufficient to relieve pain or touch sensitivity in the subject with a compromised immune system.

In all above aspects of the invention, the compositions and methods described herein also contemplate bonding the toothpaste composition (or formulations) to bristles of a toothbrush. Some embodiments include a method of bonding a flavored toothpaste composition to a substrate in the absence of a metal graft initiator, peroxide catalyst, or a combination thereof, which include the steps of providing a toothbrush having bristles comprising the substrate, bonding a monomer/prepolymer to the substrate, adding a flavoring to the monomer/prepolymer, adding a physical cross-linking polymer with the bonded monomer/prepolymer, and drying the toothbrush containing the flavored toothpaste composition.

A further embodiment is directed to a method of bonding a flavored toothpaste composition to a substrate, the substrate being the bristles of a toothbrush, which include the steps of applying a prime coat of the toothpaste composition to the bristles of the toothbrush, the prime coat including a monomer/prepolymer, flavoring and other auxiliary excipients; applying a top coat of the toothpaste composition to the prime coat, the top coat comprising a physical cross-linking polymer and other auxiliary excipients; and air drying the toothbrush containing the flavored toothpaste composition.

In some embodiments the substrate includes a functional group selected from the group consisting of amines, amides, imides, alcohols, acids, carbonyls, carboxyls, isocyanates, and combinations thereof.

In some embodiments the substrate includes nylon, polyester, or a combination thereof.

Some embodiments the monomer/prepolymer includes a polyvinyl alcohol, acrylic acid, polyacrylic acid, methacrylic acid, ethyl acrylate, ethyl methacrylate, isobutylmethacrylate, and methyl acrylate.

In some embodiments the physical cross-linking polymer is cellulose gum, carrageenan, or a combination thereof.

In some embodiments the flavoring is menthol, spearmint, or peppermint based.

In some embodiments include the optional step of curing the toothbrush containing the flavored toothpaste composition.

In some embodiments include a step of curing the toothbrush containing the flavored toothpaste composition for about 1 to 5 minutes. In further embodiments the curing is conducted for about one to about two minutes.

Some embodiments adding the flavoring to the polymer chain includes chemical bonding.

In some embodiments adding the flavoring to the polymer chain includes physical bonding.

Some embodiments include growing polymer chains on the substrate.

In another embodiment, a method is disclosed for bonding a flavored toothpaste composition to a substrate in the absence of a metal graft initiator, peroxide catalyst, or a combination thereof, wherein the method involves providing a toothbrush having bristles comprising the substrate, growing polymer chain by chemically bonding a monomer/prepolymer to the substrate, adding a flavoring to the monomer/prepolymer; and adding a physical cross-linking polymer with the bonded monomer/prepolymer, and drying the toothbrush containing the flavored toothpaste composition.

In yet another embodiment, a method is disclosed for bonding a flavored toothpaste composition to a substrate in the absence of a metal graft initiator, peroxide catalyst, or a combination thereof, wherein the method involves providing a toothbrush having bristles comprising the substrate; bonding a monomer/prepolymer to the substrate; adding a flavoring to the monomer/prepolymer; adding a physical cross-linking polymer with the bonded monomer/prepolymer, wherein the physical linking is obtained through, at least partially, intermolecular hydrogen bonding; and drying the toothbrush containing the flavored toothpaste composition.

In the compositions and methods described in all above aspects of the invention, the finished product can be sterilized and packaged to maintain sterile condition. Thus, another aspect of the invention relates to a disposable pre-pasted toothbrush package, comprising the sterilized toothbrush and toothpaste composition, described according to the above aspects of the invention, and a sealed package wrapping surrounding the toothbrush and toothpaste composition for maintaining the toothbrush and toothpaste composition in a sterile condition.

Due to their extra soft bristles, small head, fluoride- and numbing agent-containing toothpaste formulation, sterilization, and disposability, such pre-pasted toothbrushes are particularly well-suited for use by patients with a compromised immune system, because of the benefits of relieving pain or touch sensitivity, being less intrusive and provide improved comfort, and being sterile to reduce further infections in the patients.

Another aspect of the invention relates to a method of reducing pain or touch sensitivity while brushing for a subject with a compromised immune system. The method comprises providing the sterilized toothbrush and toothpaste composition, described according to the above aspects of the invention, to the subject with a compromised immune system; and applying the toothpaste composition to the subject thereby reducing pain or touch sensitivity while brushing.

### DETAILED DESCRIPTION OF THE EMBODIMENTS

The embodiments of the invention and the various features and advantages thereto are more fully explained with references to the non-limiting embodiments and examples that are described and set forth in the following descriptions of those examples. Descriptions of well-known components and techniques are omitted to avoid obscuring the invention. The examples used herein are intended merely to facilitate an understanding of ways in which the invention may be practiced and to further enable those skilled in the art to practice the invention. Accordingly, the examples and embodiments set forth herein should not be construed as limiting the scope of the invention, which is defined by the claims.

As used herein, terms such as “a,” “an,” and “the” include singular and plural referents unless the context clearly demands otherwise.

As used herein the term “about” means within 10% of a stated number.

Toothbrush and Toothpaste Composition, and Methods for Bonding Toothpaste Composition to Toothbrush Bristles Without a Metal Graft Initiator and/or Catalyst

### Chemical Grafting

In some of the contemplated embodiments, chemical grafting does not involve a metal graft initiator and/or a catalyst. Instead, carbon-carbon bonds or carbon-nitrogen bonds grow on a substrate via simple reactions, such as condensation reactions, to provide bonding to the substrate without damaging any of the existing positive characteristics of the materials involved. Such polymerizations proceed through step-by-step succession reactions between two functional groups such as amines, amides, imides, alcohols, acids, carbonyls, carboxyls, isocyanates, and other such functional groups. Each of the steps involves the elimination of two reactive sites and results in a new linking unit between a pair of molecules with the elimination of byproducts such as water, alcohol, HCl, or NaCl. For example, in the case of a nylon, crosslinking with a polymer such as polyvinyl alcohol will occur via condensation reactions between the nylon amides and multiple hydroxyl groups comprising the polyvinyl alcohol. Step-growth reactions may also proceed by polyaddition reactions without the elimination of any byproducts such as reactions between diisocyanates with diols.

Further, condensation reactions between two monomers, two oligomers, a monomer and oligomer/macromolecule, or two macromolecules are contemplated within the scope of embodiments. As such, gradual enhancement in the molar mass of the product is inevitable but the reactivity remains equal regardless of molecular weight. The structure of condensation linkages are determined by the functionality of the linking monomers or polymers and the molar ratio of the reactive sites. For example, two bifunctional monomers react to give a linear polymer but when polyfunctional monomers are involved, successively higher conversion leads to branch polymers and the probability of network formation enhances.

One of skill in the art necessarily understands reactions between functional groups that would be suitable for the purposes of crosslinking multiple polymers and/or monomers.

### Physical Cross-Linking

Physical cross-linking also affords the advantage of linking polymers with other polymers and/or monomers without the use of metal initiators and catalysts. As such the cross-linking is reversible and provides a relative ease of production when compared to chemical cross-linking and grafting techniques.

In some embodiments, physically cross-linked polymers are formed when cooling hot solutions of carrageenan. The linked network is formed due to helix-formation, association of the helices, and forming junction zones. Carrageenan in hot solution above the melting transition temperature is present as random coil conformation. Without wishing to be bound by theory, when mixed with other polymers, the random carrageenan coils physically intertwine with the other polymers. Then upon cooling the carrageenan transforms to rigid helical rods. In the presence of a salt (K⁺, Nat, etc.), due to screening the repulsion between sulphonic groups (SO₃⁻), double helices further aggregate to form a stable matrix. Consequently, the rigid helices provide a physical link with other polymers present when heated. These embodiments are also effective at entrapping other molecules (e.g. flavors).

In some embodiments, the physical cross-linking can be obtained through intermolecular hydrogen bonding. Examples include networks formed by hydrogen-bound cellulose gum (carboxymethylcellulose or CMC). In such systems, the hydrogen bonds between the carboxyl groups induce a decrease in CMC solubility in water and result in the formation of an elastic polymer matrix. A person of skill in the art necessarily understands that similar polymer systems may be formed with functional groups that provide intermolecular hydrogen bonding.

### Method of Preparation of the Mint-Flavored Toothpaste Formulation

Some embodiments are directed to a polymeric toothpaste composition that is chemically grafted to the bristles of a toothbrush to form a strong adhesion to the bristles over an extended period of time. As previously stated, chemical grafting does not involve a metal graft initiator and/or a catalyst, and instead, grows carbon-carbon bonds or carbon-nitrogen bonds on a substrate via simple reactions, such as multiple condensation reactions.

The monomers and polymers preferably comprise vinyl alcohol monomers, acrylic, or polyvinyl alcohol polymers, which chemically bond to the bristles. The monomers and prepolymers are preferably acrylic monomers having one or more hydroxyl and carboxyl groups. Some of the monomers and prepolymers of this type are the following: polyvinyl alcohol, acrylic acid, polyacrylic acid, methacrylic acid, ethyl acrylate, ethyl methacrylate, isobutylmethacrylate, and methyl acrylate. Also, mixtures of two or more monomers can be used. Polyvinyl alcohol, acrylic acid and polyacrylic acid are preferred.

The formulation is prepared from a prime coat and a top coat. The prime coat consists basically of the flavoring and the prepolymer and/or monomer, whereas the top coat consists of the polymer providing physical cross-linking properties, pigment and other auxiliary excipients.

The prime coat preferably comprises a polyvinyl alcohol, water, flavor, polysorbate 20, sodium benzoate, potassium sorbate, and methylparaben. The prime coat is applied to the bristles by dipping, then heated at an elevated temperature for 1 to 10 minutes. The amount of prepolymer and/or monomer in the prime coat will normally range from about 0.0001% to about 10.0%, though the preferred concentration ranges from about 3.0% to about 10.0% by weight.

The top coat comprises filler, namely glycerin, hydrated silica, sodium lauryl sulfate, flavor, titanium dioxide, aluminum hydroxide, sodium saccharin, polysorbate 20, trisodium ethylenediaminetetraacetic acid (EDTA), methylparaben, propylparaben, sodium benzoate, and blue 1 (Cl 42090), which are combined in a container and mixed until a uniform mix is obtained. The amount of polymer in the top coat will normally range from about 0.0001% to about 10% by weight of the composition, though the preferred concentration ranges from about 0.01% to about 1.0% by weight. A solution of the pigment is prepared, added to the uniform mix, and mixed until a uniform blend is obtained. The desired amounts of the polymers for physical cross-linkage, namely cellulose gum and carrageenan, are then added to the blend and the contents are mixed well. The prepared formulation may then be applied as a top coat over the prime coat by dipping the already flavor-treated toothbrush into the top coat and curing at 300° F. for about 2 minutes.

The amounts of the reactants employed may vary. The total amount of the polymers employed will normally range from about 0.0001% to about 10% by weight of the composition, though the preferred concentration ranges from about 0.1% to about 10% by weight, and a most preferred concentration ranges from about 1.0% to about 7.7% by weight.

Suitable materials for use as flavors are those which allow the user to detect a strong, noticeable flavor while permitting maintenance of an acceptable product appearance. The flavors may be of natural or synthetic origin. Preferably, a menthol based flavor is used. Further suitable flavors include peppermint, spearmint, wintergreen, cinnamon, and the like; fruit flavors such as cherry, strawberry, lime, and the like may also be used. Preferred flavors for use in toothpaste include peppermint, spearmint, and menthol.

Without wishing to be bound by any theory, the flavoring is physically held within a stable polymer matrix provided by a polymer from the top coat such as carrageenan. Further, in the presence of polymers such as cellulose gum, the flavoring is bound through hydrogen bonding. In some embodiments, both carrageenan and cellulose gum are implemented to hold the flavoring within the toothpaste formulation. In some embodiments menthol based flavoring may be chemically bond with the substrate, monomers, and other polymers through condensation reactions as described herein.

Examples of auxiliary excipients utilized in the present invention include fillers, surfactants, buffers and pigments.

In the preferred embodiment of the invention, the bristles of the toothbrush are dipped into a toothpaste that contains monomers, prepolymers, and other excipients of the composition. The coated samples are then air dried at room temperature for approximately 30 to 40 minutes, whereby the hydroxyl and carboxyl groups from the monomers/prepolymers in the toothpaste polymerize to form a strong adhesion to the bristles of the toothbrush.

Sterile, Disposable Toothbrush and Toothpaste Composition for Patients with a Compromised Immune System, Methods of Manufacturing Thereof, and Methods of Using Thereof

One aspect of the invention also relates to a sterilized toothbrush and toothpaste composition for a subject with a compromised immune system, comprising: a toothbrush having bristles comprising a substrate; and a toothpaste composition bonded to the substrate of the toothbrush bristles in the absence of a metal graft initiator, peroxide catalyst, or a combination thereof.

The toothpaste composition includes the flavored toothpaste formulation discussed in the aspects and embodiments above relating to the toothbrush and toothpaste composition, and methods for bonding toothpaste composition to toothbrush bristles without a metal graft initiator and/or catalyst.

In addition, the toothbrush and toothpaste composition are specifically designed for a subject with a compromised immune system, to assist proper oral care for these subjects. Examples of subjects with a compromised immune system include those cancer and transplant patients who are taking certain immunosuppressive drugs and/or chemotherapy; patients with HIV/AIDS; and patients with inherited diseases that affect the immune system, etc.

For example, patients who are receiving cancer therapy often have changes in the mouth. Chemotherapy affects rapidly dividing cells, including cancer cells and some normal cells, such as those lining the mouth. Chemotherapy cannot tell the difference between normal cells and malignant cells and sometimes injures both, therefore making cancer patients more at risk for infection and/or bleeding. If there is pre-existing dental infection such as cavities, abscesses, or gum (periodontal) disease, the infection may become worse. Chemotherapy patients' gums may bleed easily if they are irritated or swollen. Chemotherapy patients' also often have extreme sensitivity in his mouth, both to the teeth and gums.

In addition, chemotherapy may also cause oral sores (mucositis), painful ulcers of the oral mucosa, which can interferes with proper nutrition and hydration of cancer patients, further undermining patents' health. These ulcers also present an increased risk of infection as more serious ulcers may become infected with bacteria or yeast that are commonly found in the mouth.

Therefore, the toothbrush and toothpaste composition for a subject with a compromised immune system also comprise an anti-cavity agent to address pre-existing dental infection or prevent future dental infection such as cavities.

A typical anti-cavity agent is fluoride, for instance, as a soluble fluoride salt, e.g., sodium fluoride, stannous fluoride, or sodium monofluorophosphate. In one embodiment, the anti-cavity agent is sodium fluoride. The anti-cavity agent is used in an effective amount to prevent cavities. In the case of fluoride, the amount in the toothpaste composition typically ranges from about 250 to about 25,000 ppm fluoride ions, for instance, from about 500 to about 2,500 ppm fluoride ions, or from about 1,000 to about 2,500 ppm fluoride ions. For children, the amount of fluoride ions in the toothpaste composition typically ranges from about 500 to about 2,500 ppm, or from about 500 to about 1,500 ppm. For adults, the amount of fluoride ions in the toothpaste composition typically ranges from about 1,000 to about 2,500 ppm, or from about 1,000 to about 1,500 ppm.

The toothbrush and toothpaste composition for a subject with a compromised immune system also comprise a numbing agent in an amount sufficient to relieve pain or touch sensitivity in the subject, to allow for pain-free brushing. Suitable numbing agents include, but are not limited to, eugenol, benzocaine, and lidocaine. In one embodiment, the numbing agent is eugenol. The amount of the numbing agent, e.g., eugenol, may be present in the toothpaste composition in an amount ranges from about 0.001 wt % to about 2 wt %, for instance, from about 0.01 wt % to about 1 wt %, or from about 0.05 wt % to about 0.5 wt %.

The toothbrush is made with extra soft bristles and with small, compact bristle head to make the toothbrush more gentle and less intrusive and cause minimum oral irritation while brushing.

In some embodiments, the substrate of the toothbrush bristles is formed from a soft, flexible elastomer selected from the group consisting of nylon, polypropylene, polyester, and combinations thereof. Typically, the substrate of the toothbrush bristles is formed from nylon.

In some embodiments, the shape of the ends of bristles is feathered (splayed) to make the bristles even softer. The touch-points of bristles made from this shape are gentle and do not damage or injure teeth, gums and other soft tissue of the mouth during brushing.

The dimensions of the tooth bristles are small enough to fit in a small and compact head. In some embodiments, the length of each toothbrush bristle ranges from about 10 mm to about 15 mm, for instance, from about 10 mm to about 12 mm. In some embodiments, the diameter of a toothbrush bristle ranges from about 0.10 mm to about 0.2 mm, for instance, from about 0.10 mm to about 0.15 mm.

The extra soft bristles that are fit in a small compact head therefore provide better comfort and functionality for chemotherapy patients that have extreme sensitivity in their mouth.

The finished toothbrush and toothpaste composition are sterilized with an ozone treatment to maintain the sterile conditions of the toothbrush to avoid further infections.

Another aspect of the invention relates to a method of manufacturing a sterilized toothbrush and toothpaste composition for a subject with a compromised immune system, comprising: providing a toothbrush having bristles comprising a substrate; bonding a toothpaste composition to the substrate of the toothbrush bristles in the absence of a metal graft initiator, peroxide catalyst, or a combination thereof; and sterilizing the toothbrush and toothpaste composition.

As discussed in the above aspect of the invention, the toothpaste composition includes the flavored toothpaste formulation discussed in the aspects and embodiments above relating to the toothbrush and toothpaste composition, and methods for bonding toothpaste composition to toothbrush bristles without a metal graft initiator and/or catalyst.

The toothpaste composition also comprises an anti-cavity agent and a numbing agent in an amount sufficient to relieve pain or touch sensitivity in the subject with a compromised immune system.

Suitable anti-cavity agents and the amounts used in the toothpaste composition are the same as those described above in the aspect of the invention relating to a sterilized toothbrush and toothpaste composition for a subject with a compromised immune system.

In one embodiment, the anti-cavity agent is sodium fluoride.

Suitable numbing agents and the amounts used in the toothpaste composition are the same as those described above in the aspect of the invention relating to a sterilized toothbrush and toothpaste composition for a subject with a compromised immune system.

In some embodiments, the numbing agent is eugenol, benzocaine, or lidocaine. In one embodiment, the numbing agent is eugenol.

Suitable toothbrush and bristle materials and the dimensions and shapes are the same as those described above in the aspect of the invention relating to a sterilized toothbrush and toothpaste composition for a subject with a compromised immune system.

In some embodiments, the substrate of the toothbrush bristles is formed from a soft, flexible elastomer material selected from the group consisting of nylon, polypropylene, polyester, and combinations thereof. In one embodiment, the substrate of the toothbrush bristles is formed from nylon.

In one embodiment, the shape of the ends of bristles is feathered.

In some embodiments, the length of each toothbrush bristle ranges from about 10 mm to about 15 mm, for instance, from about 10 mm to about 12 mm. In some embodiments, the diameter of a toothbrush bristle ranges from about 0.10 mm to about 0.2 mm, for instance, from about 0.10 mm to about 0.15 mm.

The sterilization process can be carried out by an ozone treatment, which includes air-spraying the toothbrush and toothpaste composition with an ozone gas or an aqueous ozone to coat the toothbrush and toothpaste composition with ozone. The ozone serves as a disinfectant that can eliminate microbes such as viruses and bacteria and maintain the sterile conditions of the toothbrush and toothpaste composition. The excess ozone can break down to oxygen and dissipate into the air.

Alternatively, the sterilization process can be carried out by gamma ray radiation, which includes subjecting the toothbrush and toothpaste composition to gamma ray radiation at a sufficient intensity and time to sterilize the toothbrush and toothpaste composition.

The method can further comprise individually sealing (e.g., by hermetically sealing) the sterilized toothbrush and toothpaste composition with a package to wrap surrounding the toothbrush and toothpaste composition for maintaining the toothbrush and toothpaste composition in a sterile condition.

Thus, another aspect of the invention relates to a disposable pre-pasted toothbrush package, comprising the sterilized toothbrush and toothpaste composition, described according to the above aspects of the invention, and an individually sealed package wrapping surrounding the toothbrush and toothpaste composition for maintaining the toothbrush and toothpaste composition in a sterile condition.

All above descriptions and all embodiments regarding the toothbrush, toothbrush bristles, toothpaste composition, and sterilization discussed above in the aspect of the invention relating to a sterilized toothbrush and toothpaste composition for a subject with a compromised immune system are applicable to this aspect of the invention.

In some aspects, the invention also relates to a method of reducing pain or touch sensitivity while brushing for a subject with a compromised immune system. The method comprises providing the sterilized toothbrush and toothpaste composition, described according to the above aspects of the invention, to the subject with a compromised immune system; and applying the toothpaste composition to the subject thereby reducing pain or touch sensitivity while brushing.

All above descriptions and all embodiments regarding the toothbrush, toothbrush bristles, toothpaste composition, and sterilization discussed above in the aspect of the invention relating to a sterilized toothbrush and toothpaste composition for a subject with a compromised immune system are applicable to this aspect of the invention.

Certain features and aspects of the present invention are illustrated in the following working examples. The working examples are merely exemplary which are strictly demonstrative and not to be construed as limiting in scope.

### Example 1

TABLE 1
Prime Coat

| Ingredient | % Weight |
|---|---|
| Water | 91.15000 |
| Polyvinyl Alcohol | 7.00000 |
| Flavor/Aroma | 1.00000 |
| Polysorbate 20 | 0.10000 |
| Sodium Benzoate | 0.30000 |
| Potassium Sorbate | 0.20000 |
| Limonene | Part of Flavor |
| Methylparaben | 0.25000 |
| Total | 100.00000 |

TABLE 2
Top Coat

| Ingredient | % Weight |
|---|---|
| Water | 40.96727 |
| Glycerin | 35.64000 |
| Hydrated Silica | 16.00000 |
| Sodium Lauryl Sulfate | 2.80000 |
| Flavor/Aroma | 2.00000 |
| Cellulose Gum | 0.40000 |
| Carrageenan | 0.14100 |
| Titanium Dioxide | 0.30000 |
| Aluminum Hydroxide | 0.50000 |
| Sodium Saccharin | 0.50000 |
| Polysorbate 20 | 0.15000 |
| Trisodium EDTA | 0.10000 |
| Methylparaben | 0.25000 |
| Propylparaben | 0.15000 |
| Sodium Benzoate | 0.10000 |
| Cinnamal | Part of Flavor |
| Limonene | Part of Flavor |
| Total | 100.00000 |

In the above exemplary toothpaste formulations, fluoride is added to improve efficacy of the toothpaste, and a numbing agent (benzocaine, lidocaine, or eugenol) is added to relieve pain or touch sensitivity. Table 3 below shows an exemplary toothpaste formulation for a subject with a compromised immune system.

TABLE 3
Exemplary toothpaste formulation

| Ingredient | % Weight |
|---|---|
| Sodium Fluoride | 0.25 |
| Water | ~40 |
| Glycerin | ~35 |
| Hydrated Silica | ~16 |
| Sodium Lauryl Sulfate | ~3 |
| Flavor/Aroma | ~2 |
| Carboxymethyl Cellulose | <1 |
| Hydrolyzed Gardenia Florida Extract | <1 |
| Stevia Rebaudina Leaf Extract | <1 |
| Carrageenan | <1 |
| Titanium Dioxide | <1 |
| Aluminum Hydroxide | <1 |
| Polysorbate 20 | <1 |
| Trisodium EDTA | <1 |
| Methylparaben | <1 |
| Propylparaben | <1 |
| Sodium Benzoate | <1 |
| Sucralose Powder | <1 |
| Maltodextrin | <1 |
| Eugenol | <1 |
| Xylitol | <1 |
| Total | 100 |

The bristles are manufactured from a thin nylon material with a dimension of about 10 mm to about 15 mm in length, and about 0.10 mm to about 0.15 mm in diameter to be extra soft, and disposed on a small bristle head. The finished pre-pasted toothbrush products are packaged, placed on pallets, and then undergo a sterilization process which can be on-site at the manufacturing facility, or at a remote location. The sterilization process involves air-spraying the toothbrush and toothpaste composition with an ozone gas to coat the toothbrush and toothpaste composition with ozone. The sterilized toothbrush and toothpaste composition is then individually sealed hermetically with a package to wrap around the toothbrush and toothpaste composition, which then maintains the toothbrush and toothpaste composition in a sterile condition.

What is claimed is:

1. A sterilized toothbrush and toothpaste composition for a subject with a compromised immune system, comprising:
(a) a toothbrush having bristles comprising a substrate; and
(b) a toothpaste composition bonded to the substrate of the toothbrush bristles in the absence of a metal graft initiator, peroxide catalyst, or a combination thereof, wherein the toothpaste composition comprises:
an anti-cavity agent, and
a numbing agent in an amount sufficient to relieve pain or touch sensitivity in the subject with a compromised immune system;
wherein the toothbrush and toothpaste composition are sterilized with an ozone treatment.

2. The sterilized toothbrush and toothpaste composition of claim 1, wherein the substrate of the toothbrush bristles is formed from a soft, flexible elastomer selected from the group consisting of nylon, polypropylene, polyester, and combinations thereof.

3. The sterilized toothbrush and toothpaste composition of claim 1, wherein the substrate of the toothbrush bristles is formed from nylon.

4. The sterilized toothbrush and toothpaste composition of claim 1, wherein the shape of the ends of bristles is feathered.

5. The sterilized toothbrush and toothpaste composition of claim 1, wherein the length of each toothbrush bristle ranges from about 10 mm to about 12 mm; and the diameter of a toothbrush bristle ranges from about 0.10 mm to about 0.15 mm.

6. The sterilized toothbrush and toothpaste composition of claim 1, wherein the anti-cavity agent is sodium fluoride.

7. The sterilized toothbrush and toothpaste composition of claim 1, wherein the numbing agent is eugenol, benzocaine, or lidocaine.

8. The sterilized toothbrush and toothpaste composition of claim 7, wherein the numbing agent is eugenol.

9. A disposable pre-pasted toothbrush package, comprising:
the sterilized toothbrush and toothpaste composition of claim 1, and
a sealed package wrapping surrounding the toothbrush and toothpaste composition for maintaining the toothbrush and toothpaste composition in a sterile condition.

10. A method of manufacturing a sterilized toothbrush and toothpaste composition for a subject with a compromised immune system, comprising:
providing a toothbrush having bristles comprising a substrate;
bonding a toothpaste composition to the substrate of the toothbrush bristles in the absence of a metal graft initiator, peroxide catalyst, or a combination thereof, wherein the toothpaste composition comprises an anti-cavity agent and a numbing agent in an amount sufficient to relieve pain or touch sensitivity in the subject with a compromised immune system; and
sterilizing the toothbrush and toothpaste composition.

11. The method of claim 10, further comprising:
sealing the sterilized toothbrush and toothpaste composition with a package to wrap surrounding the toothbrush and toothpaste composition for maintaining the toothbrush and toothpaste composition in a sterile condition.

12. The method of claim 10, the substrate of the toothbrush bristles is formed from a soft, flexible elastomer selected from the group consisting of nylon, polypropylene, polyester, and combinations thereof.

13. The method of claim 12, wherein the substrate of the toothbrush bristles is formed from nylon.

14. The method of claim 13, wherein the shape of the ends of bristles is feathered.

15. The method of claim 10, wherein the length of each toothbrush bristle ranges from about 10 mm to about 12 mm; and the diameter of a toothbrush bristle ranges from about 0.10 mm to about 0.15 mm.

16. The method of claim 10, wherein the anti-cavity agent is sodium fluoride.

17. The method of claim 10, wherein the numbing agent is eugenol, benzocaine, or lidocaine.

18. The method of claim 17, wherein the numbing agent is eugenol.

19. The method of claim 10, wherein the sterilizing is carried out by air-spraying the toothbrush and toothpaste composition with ozone gas to coat the toothbrush and toothpaste composition with ozone.

20. A method of reducing pain or touch sensitivity while brushing for a subject with a compromised immune system, comprising:
providing the sterilized toothbrush and toothpaste composition according to claim 1 to the subject with a compromised immune system; and
applying the toothpaste composition to the subject thereby reducing pain or touch sensitivity while brushing.
